SEQUENCE LISTING (15 sequences)

SEQ ID NO: 1 - protein, 16 aa, Artificial Sequence
  SV40 large T antigen nuclear localization signal peptide
PPKKKRKVPPKKKRKV

SEQ ID NO: 2 - protein, 19 aa, Artificial Sequence
  endoplasmic reticulum-targeting signal peptide
KDELAKDELAKDELAKDEL

SEQ ID NO: 3 - protein, 28 aa, Artificial Sequence
  cytochrome C oxidase mitochondrial-targeting signal peptide
SVTTPLLLRGLTGSARRLPVPRAKIHSL

SEQ ID NO: 4 - protein, 16 aa, Artificial Sequence
  PTS1 peroxisome-targeting signal peptide
SKLASKLASKLASKLA

SEQ ID NO: 5 - protein, 11 aa, Artificial Sequence
  HIV Tat protein nuclear localization signal peptide
YGRKKRRQRRR

SEQ ID NO: 6 - protein, 19 aa, Artificial Sequence
  cell membrane-targeting signal peptide
KLNPPDESGPCMSCKCVLS

SEQ ID NO: 7 - protein, 20 aa, Artificial Sequence
  GAP-43 cell membrane-targeting signal peptide
MLCCMRRTKQVEKNDEDQKI

SEQ ID NO: 8 - protein, 16 aa, Artificial Sequence
  ARF-1 trans-Golgi body-targeting signal peptide
MXXEMXXEMXXEMXXE

SEQ ID NO: 9 - protein, 9 aa, Artificial Sequence
  epidermal growth factor receptor (EGFR) targeting cell surface receptor signal peptide
EEEEYFELV

SEQ ID NO: 10 - protein, 13 aa, Artificial Sequence
  adenovirus nuclear localization signal peptide
CGGFSTSLRARKA

SEQ ID NO: 11 - protein, 15 aa, Artificial Sequence
  integrin targeting cell surface receptor signal peptide
CKKKKKKGGRGDMFG

SEQ ID NO: 12 - protein, 12 aa, Artificial Sequence
  Rac1 phosphatidylinositol 4,5-phosphate kinase (PIP5K) kinase targeting signal peptide
CPPPVICKKRKR

SEQ ID NO: 13 - protein, 7 aa, Artificial Sequence
  alpha(IIb)beta(3) integrin targeting signal peptide
KVGFFKR

SEQ ID NO: 14 - protein, 16 aa, Artificial Sequence
  biotinylated SV40 large T antigen nuclear localization signal peptide, biotinylated nuclear localization signal (NLS) sequence
PPKKKRKVPPKKKRKX

SEQ ID NO: 15 - protein, 16 aa, Artificial Sequence
  biotinylated random peptide sequence
XPPLSQEAFADLLKKK

(19) United States
(12) Patent Application Publication
(10) Pub. No.: US 2008/0118941 A1
(43) Pub. Date: May 22, 2008

(54) Signal Peptide-Semiconductor Nanocrystal Conjugates

(75) Inventors: Fanqing Chen, Moraga, CA (US); Daniele Gerion, Albany, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/661,946

(22) PCT Filed: Sep. 2, 2005
(86) PCT No.: PCT/US05/31386

§ 371 Date: Mar. 2, 2007

Related U.S. Application Data
(60) Provisional application No. 60/607,286, filed on Sep. 2, 2004.

(51) Int. Cl. C12Q 1/02; C07K 7/00; C07K 14/00; G01N 33/58; C12N 5/06; C07K 2/00; C07K 7/08

(52) U.S. Cl. 435/29; 530/300; 530/324; 530/327; 530/328; 530/329; 530/326; 435/7.8; 977/813; 435/375; 977/795

(57) ABSTRACT

The present invention provides signal peptide-semiconductor nanocrystal-peptide conjugates and methods for using the conjugates in methods for imaging live cells and subcellular trafficking processes.

### CROSS-REFERENCES TO RELATED APPLICATIONS

This application claims benefit of U.S. Provisional Application No. 60/607,286, filed Sep. 2, 2004, which application is incorporated herein by reference for all purposes.

### STATEMENT AS TO RIGHTS TO INVENTIONS MADE UNDER FEDERALLY SPONSORED RESEARCH OR DEVELOPMENT

This work was supported by NIH Grant R21CA95393-01, by NASA grant NNA04CA75I, and by the U.S. Department of Energy at Lawrence Berkeley National Laboratory under contract no. DE-AC03-76SF00098, now contract no. DE-AC02-05-CH11231, and at Lawrence Livermore National Laboratory under contract No. W-7405-ENG-48. The government has certain rights in this invention.

### FIELD OF THE INVENTION

This invention relates to the field of semiconductor nanocrystals and to the field of imaging and monitoring live cells, cellular behavior and phenomena.

### BACKGROUND OF THE INVENTION

To understand the complexity and dynamics of cellular events in living organisms, it is desirable to image the nucleic acids, proteins, or metabolites inside living cells. In live cell imaging, the entry of the probe into the nucleus and its visualization constitute increasingly important areas of research (Jans et al., Medicinal Research Reviews 18:189-223 (1998); Jans et al., Bioessays 22:532-544 (2000)). The nucleus is a desirable target because the genomic DNA, which carries the genetic information of the cell, resides there. In addition, numerous nuclear proteins participate actively in critical cellular processes, such as DNA replication, recombination, RNA transcription, DNA damage and repair, genomic alterations, and cell cycle control. The efficient transport of probes into the nuclei of living cells would greatly enhance the diagnosis of disease genotype, the tracking of oligonucleotide and other nucleus-targeting drugs, the understanding of biological processes in the nucleus, and the identification of potential nuclear drug candidates. However, in living cells, a double-membrane nuclear envelope separates the cytoplasm from the cell nucleus. This physical barrier is impermeable to most kinds of probes, except at specific locations, a few tens of nanometers wide, called the nuclear pores (see, e.g., Fahrenkrog et al., Nature Reviews Molecular Cell Biology, 4:757-766 (2003)).

Currently, for imaging living cells, fluorescent tagging with organic fluorophores (Swedlow et al., Cell Structure and Function, 27:335-341 (2002)) or green fluorescent protein (GFP) (Ehrhardt, Curr. Op. Plant Biol. 6:622-628 (2003)) is still the most commonly used method. Unfortunately, organic dyes are usually toxic to the cells and therefore the use of organic fluorophores for live cell applications has obvious limitations. Moreover, organic dyes and GFP both suffer from notorious shortcomings such as photobleaching, which preclude their use in many long-term imaging applications. These fluorophores also have limited sensitivity and resolution, both of which are critical factors for accurate tracking of individual biomolecules. Finally, recombinant GFP fusion proteins are cumbersome to construct, and long-term imaging (>3 days) with GFP requires the time-consuming process of establishing stable-expressing clones.

To solve the stability and sensitivity issue, other types of labels such as polymeric (Liu et al., Biomacromolecules, 2:362-368 (2001)), magnetic (Hinds et al., Blood, 102:867-872 (2003)) and metallic (Cognet et al., PNAS USA 100:11350-11355 (2003); Tkachenko et al., J. Amer. Chem. Soc., 125:4700-4701 (2003); Schultz et al., PNAS USA 97:996-1001 (2000)) particles have been introduced into cells. However, fluorescence microscopy remains the simplest and most-used detection tool, and it would therefore be desirable to develop a technology based on robust fluorescent probes. Inorganic semiconductor nanocrystals, or qdots, represent this alternative technology (Bruchez, Jr. et al., Science, 281:2013-2016 (1998)). Semiconductor nanocrystals, such as CdSe/ZnS core/shell nanoparticles, are inorganic fluorophores with a size below 10 nm. Compared to conventional dyes, they have a much higher photobleaching threshold and negligible photobleaching under biological imaging conditions. Semiconductor nanocrystals can be silanized as described by Gerion et al., J. Phys. Chem., 105:8861-8871 (2001). Silanized semiconductor nanocrystals have reduced phototoxicity and are highly resistant to chemical and metabolic degradation in vivo (Pellegrino et al., Differentiation, 71:542-548 (2003)). Finally, whereas organic fluorophores require customized chemistry for conjugation of biomolecules to each fluorophore, a universal approach can be used for the conjugation of biomolecules to all silanized semiconductor nanocrystals, because the silica shell coatings for different semiconductor nanocrystals are identical.

This technology is progressing at a fast pace (Alivisatos, Nature Biotechnol., 22:47-52 (2004); Dahan et al., Science 302:442-445 (2003)). Recently, a wide variety of biomolecules such as DNA (Gerion et al., Anal. Chem., 75:4766-4772 (2003); Dubertret et al., Science, 298:1759-1762 (2002)), proteins, antibodies (Jaiswal et al., Nat. Biotechnol., 21:47-51 (2003); Wu et al., Nat. Biotechnol., 21:41-46 (2003)), short peptides (Akerman et al., PNAS USA 99:12617-12621 (2002)), and neurotransmitters (Rosenthal et al., J. Amer. Chem. Soc. 124:4586-4594 (2002)) have been attached to semiconductor nanocrystals. For instance, semiconductor nanocrystals have been used extensively as immunohistochemical labels in fixed cells. In vitro, semiconductor nanocrystals conjugated to immunoglobin G (IgG) have been used for the detection of membrane proteins such as the cancer marker Her2. The study of living cells presents an additional difficulty, viz., the introduction of the semiconductor nanocrystals inside the cells. Different methods have been reported. Living cells moving over a qdot-coated collagen surface engulf nanoparticles, thus permitting study of their motility patterns (Pellegrino et al., Differentiation, 71:542-548 (2003)). Microinjection into Xenopus embryo has been used to follow cell dynamics during embryogenesis (Dubertret et al., Science, 298:1759-1762 (2002)). Finally, receptor-mediated endocytosis has also been used to transfect living cells. For instance, semiconductor nanocrystals bearing Epidermal Growth Factor (EGF) have been demonstrated to bind to erbB/Her receptors and are actively endocytosed into endosomes in living cells (Lidke et al., Nat. Biotechnol., 22:198-203 (2004)).

The ability to control the growth conditions, shape and size allows one to tailor and control the optical properties of semiconductor nanocrystals. Even though in vitro and in vivo imaging with semiconductor nanocrystals has been demonstrated, most of these studies have focused on the entry of qdots into the cytoplasm or targeting of the membrane proteins (Dahan et al., Science, 302:442-445 (2003); Dubertret et al., Science, 298:1759-1762 (2002); Jaiswal et al., Nat. Biotechnol., 21:47-51 (2003); Wu et al., Nat. Biotechnol., 21:41-46 (2003); Rosenthal et al., J. Amer. Chem. Soc. 124:4586-4594 (2002)). Others have used peptides conjugated to semiconductor nanocrystals to target specific tissues such as endothelial cells or carcinogenic cells (see, e.g., Akerman et al. PNAS USA 20:12617-21 (2002)).

Detection of nuclear proteins has been reported only in fixed cells by using anti-nuclear antigens (Wu et al., Nat. Biotechnol., 21:41-46 (2003)). Semiconductor nanocrystals have also been shown to accumulate in cell nucleus by passive diffusion after cell division (Dubertret et al., Science, 298:1759-1762 (2002)). However, so far, no report has investigated the feasibility of active and targeted localization of semiconductor nanocrystals into the nuclei of living cells.

The challenges for the use of semiconductor nanocrystals for targeted nuclear delivery are multiple (Jans et al., Bioessays, 22:532-544 (2000); Tkachenko et al., J. Amer. Chem. Soc. 125:4700-4701 (2003); Suh et al., PNAS USA 100:3878-3882 (2003); Goldfarb et al., Nature, 322:641-644 (1986)). First, semiconductor nanocrystals must have a surface chemistry that allows their escape from endosomal/lysosomal pathways in living cells. Second, semiconductor nanocrystals must possess a nuclear localization signal (NLS) in order to be transported by the nuclear trafficking proteins and to interact with the nuclear pore complex. Third, the diameter of the nuclear pore complex is 20-50 nm depending on the cell line (Fahrenkrog et al., Nature Reviews Molecular Cell Biology, 4:757-766 (2003)), and therefore the qdot conjugates have to be small enough (<20 nm) to cross the nuclear membrane. In addition, the qdot conjugates must enter the cell via transfection or receptor-mediated endocytosis rather than through microinjection, so that a significant number of cells can be studied. Finally, qdot conjugates must not interfere with normal physiology of the cells. Thus, there is a need in the art for nanoparticle conjugates that can specifically target cells of interest or subcellulular compartments (e.g., nuclei) within such cells. The present invention satisfies these and other needs.

### SUMMARY OF THE INVENTION

The present invention provides signal peptide-nanoparticle conjugates with a diameter of less than about 20 nm and methods of using such conjugates.

One embodiment of the invention provides a signal peptide-semiconductor nanocrystal conjugate of less than about 20 nm in diameter and comprising a semiconductor nanocrystal and a signal peptide attached to the nanocrystal to form a signal peptide-semiconductor nanocrystal conjugate. The conjugate may be about 5 to about 20 or about 10 to about 15 nm in diameter. The signal peptide may be selected from: a nuclear-localizing signal peptide, a peroxisome-targeting signal peptide, a cell membrane-targeting signal peptide, a mitochondrial-targeting signal peptide, an endoplasmic reticulum-targeting signal peptide, and a trans-Golgi body-targeting signal peptide. In a preferred embodiment, the signal peptide is a nuclear localizing signal peptide. The signal peptide may comprise a sequence with 75%, 80%, 85%, 90%, 95%, 96%, 98%, 99%, or 100% identity to a sequence selected from: SEQ ID NOS: 1-13. The signal peptide may comprise SEQ ID NO: 1. In some embodiments, the semiconductor nanocrystal comprises a core and a shell around the core. In some embodiments, the core comprises two or more elements independently selected from: Group II elements, Group III elements, Group IV elements, Group V elements, Group VI elements, and combinations thereof. In some embodiments, the shell comprises at least one material selected from: MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, GaAs, InGaAs, InP, and InAs. In some embodiments, the shell further comprises a hydrophilic coating (e.g., SiO, SiO₂, polyethylene glycol, an ether, a mercapto acid, a hydrocarbonic acid, and combinations thereof). In some embodiments, the core comprises CdSe; the shell comprises ZnS, and the hydrophilic coating comprises SiO₂. The semiconductor nanocrystal and the signal peptide may be attached via a linking agent (e.g., a linking agent that is covalently bound to the shell). The linking agents may be selected from a negatively charged moiety, a positively charged moiety, a steric repulsion group, a thiol, an amine, a carboxyl, or a polyethylene glycol. The linking agent may be a bifunctional crosslinker (e.g., a bifunctional crosslinker comprising two reactive groups independently selected from: thiol, carboxylate, carbonyl, amine, hydroxyl, aldehyde, ketone, active hydrogen, ester, sulfhydryl and a photoreactive moiety). The semiconductor nanocrystal and the signal peptide may be attached via a first linking agent and a second linking agent. The semiconductor nanocrystal may be bound to the first linking agent and the signal peptide may be bound to the second linking agent. The first linking agent and the second linking agent may be independently selected from avidin, streptavidin, biotin and a bifunctional crosslinker. In some embodiments, the first linking agent is biotin and the second linking agent is streptavidin.

A further embodiment of the invention provides methods for imaging cellular structures of a cell (e.g., subcellular compartments). A cell is contacted with the signal peptide-semiconductor nanocrystal conjugate of the invention under conditions such that the signal peptide-semiconductor nanocrystal conjugate is taken up by the cell; and the cell is imaged to track movement of the signal peptide-semiconductor nanocrystal conjugates within the cell. The cell may be a live cell. The cell may be contacted with the signal peptide-semiconductor nanocrystal conjugate for at least about 1 hour, 2, 4, 8, 12, 16, 20, 24, 36, 48, or more hours. In some embodiments, the cell is also subjected to electroporation (i.e., before, after or during contacting the cell with the signal peptide-semiconductor nanocrystal conjugate).

Another embodiment of the invention provides a method for obtaining a population of labeled cells containing signal peptide-semiconductor nanocrystal conjugates. The cell is contacted with the signal peptide-semiconductor nanocrystal conjugate under conditions such that the signal peptide-semiconductor nanocrystal conjugate is taken up by the cell. The cell is allowed to divide at least once under conditions such that, following division, each cell contains at least one signal peptide-semiconductor nanocrystal conjugate, thereby generating a population of labeled cells containing signal peptide-semiconductor nanocrystal conjugates. The cell may be contacted with the signal peptide-semiconductor nanocrystal conjugate for at least about 1 hour, 2, 4, 8, 12, 16, 20, 24, 36, 48, or more hours. In some embodiments, the cell is also subjected to electroporation (i.e., before, after or during contacting the cell with the signal peptide-semiconductor nanocrystal conjugate).

### BRIEF DESCRIPTION OF THE DRAWINGS

FIG. 1 depicts signal peptide-nanoparticle conjugates described herein and depicts from left to right: silanized semiconductor nanocrystals, streptavidin-semiconductor nanocrystals conjugates (STV-qdot), and semiconductor nanocrystals conjugated to a signal peptide through a streptavidin-biotin bridge.

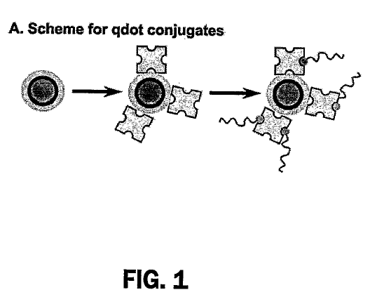

FIG. 2 depicts a silanized semiconductor nanocrystal.

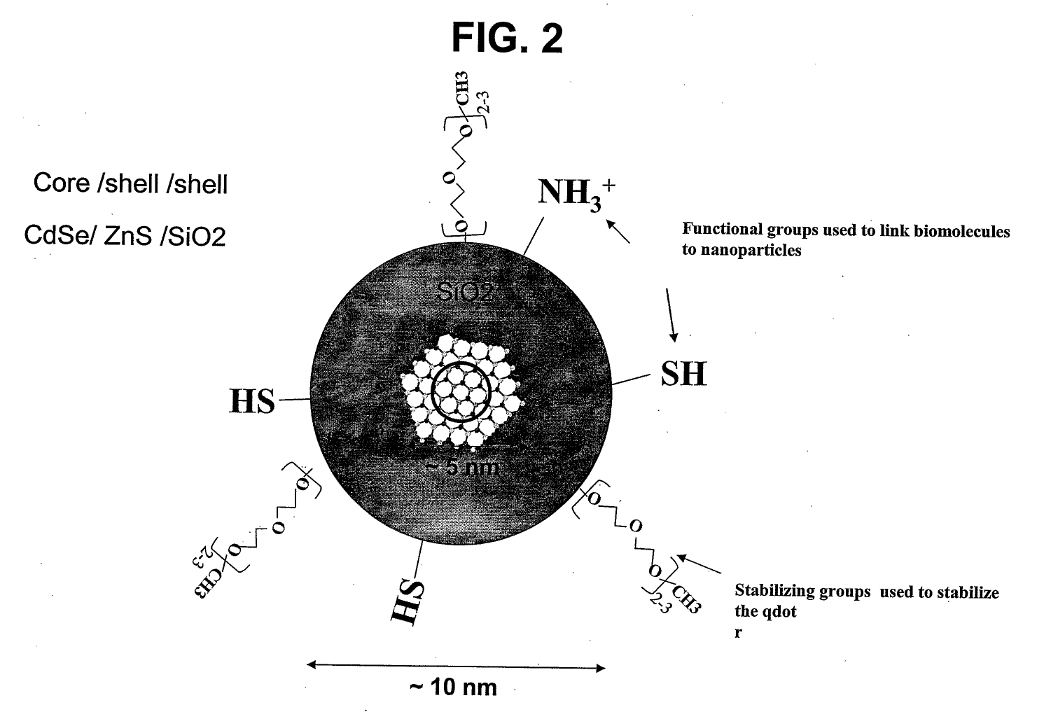

FIG. 3 depicts a signal peptide-nanoparticle conjugate wherein the signal peptide is conjugated to the nanoparticle via a covalent bond using a bifunctional crosslinker.

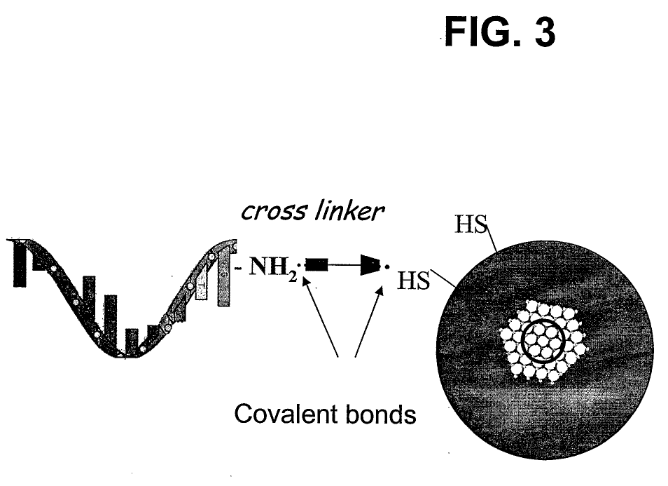

FIG. 4 depicts a signal peptide-nanoparticle conjugate wherein the signal peptide is conjugated to the nanoparticle via a binding pair such as streptavidin and biotin.

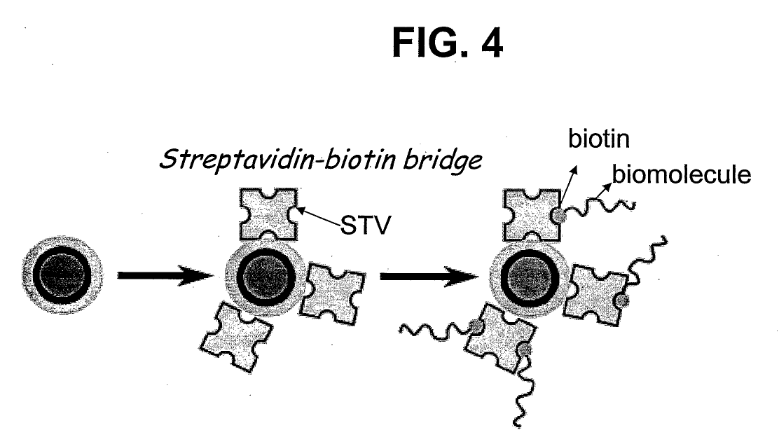

FIG. 5 depicts a signal peptide-nanoparticle conjugate wherein the signal peptide is conjugated to the nanoparticle via a heterobifunctional crosslinker comprising an amine group and a thiol group that form a heterocyclic ring for conjugation to an aldehyde group on a signal peptide.

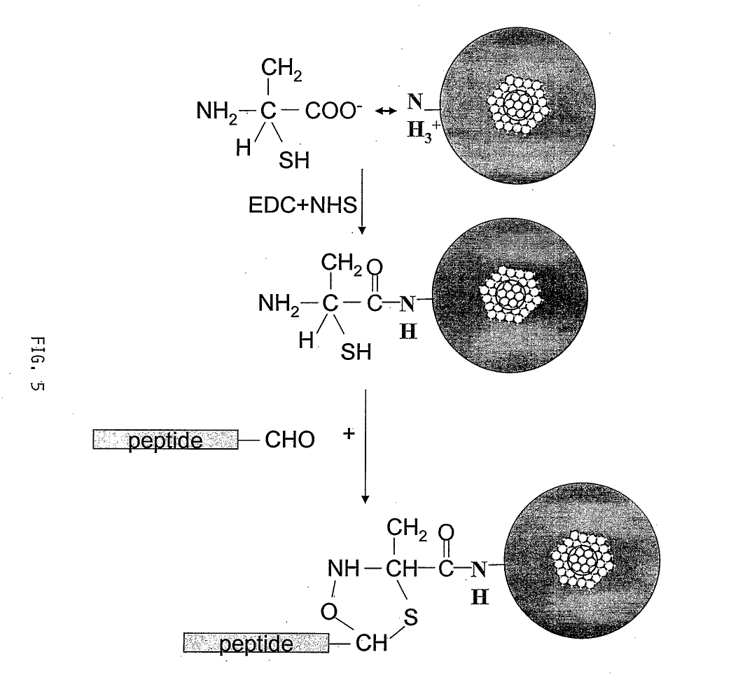

### DETAILED DESCRIPTION OF THE INVENTION

### I. Introduction

The present invention provides signal peptide-nanoparticle conjugates of less than about 20 nm in diameter. The conjugates comprise a nanoparticle (e.g. a semiconductor nanocrystal) and a signal peptide attached to the nanoparticle (i.e., covalently or via a linker). The conjugates can be used to specifically target cells or subcellular compartments within cells.

The signal peptide-nanoparticle conjugates of the invention have the advantage of low cytotoxicity, higher sensitivity, desirable photostability, long-term biological stability, resistance to lysosomal degradation, and reasonable resistance to aggregation within the cell compared to other labels (e.g., optical dyes) used to image cells. The signal peptide-nanoparticle conjugates of the invention also, surprisingly, do not interfere with the growth or differentiation of the cells. Thus, the signal peptide-semiconductor nanocrystal conjugates of the invention can be introduced into cells and retained for extended periods of time.

In contrast to dyes, which have narrow wavelength bands of absorption (e.g., about 30-50 nm) and broad wavelength bands of emission (e.g., about 100 nm) and broad tails of emission (e.g., another 100 nm) on the red side of the spectrum, semiconductor nanocrystals are capable of absorbing and emitting radiation (i.e., luminescing) in response to a broad range of wavelengths, including the range from gamma radiation to microwave radiation. The semiconductor nanocrystals are also capable of emitting radiation within a narrow wavelength band of about 50, 40, 30, 20, or 10 nm or less. Thus, a single energy source can be used to excite the luminescence of a plurality of signal peptide-semiconductor nanocrystal conjugates, each of which comprise a different semiconductor material. In addition, the plurality of signal peptide-semiconductor nanocrystal conjugates can easily be distinguished following excitation because each semiconductor nanocrystal will emit only a narrow wavelength band.

In some embodiments the signal peptide-nanoparticle conjugates are used to target subcellular compartments and organelles (including, e.g., the endoplasmic reticulum, Golgi, membrane, proteasome, perixisome, and/or mitochondria) including, for example, in methods of imaging subcellular compartments of cells and trafficking of compounds through the compartments, in methods of imaging different cell types in a single sample, or in methods of identifying cell populations that differentially express a particular marker (e.g., a cell surface marker or a transmembrane protein). The conjugates can also be used in cell motility and migration assays and in methods of delivery compounds to cells or subcellular compartments. In a particularly preferred embodiment, the peptide-semiconductor nanocrystal conjugates are capable of actively translocating to the cell nucleus.

In some embodiments of the invention, the signal peptide-semiconductor nanocrystal conjugates can be used to study cellular pathways and behavior such as the protein trafficking process, uptake, absorption, division, phagocystosis, degradation and cell death. The invention contemplates using peptide-semiconductor nanocrystal conjugates to visualize long-term biological events that occur in the cell nucleus and other subcellular compartments and structures. The invention further provides a nontoxic, long-term imaging platform for observing nuclear trafficking mechanisms and cell nuclear processes.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y.), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The term “nanoparticle” as used herein refers to a particle whose size is measured in nanometers. Nanoparticles include, e.g., semiconductor nanocrystals, metal nanocrystals, hollow nanoparticles, carbon nanospheres. The nanoparticles typically have a diameter in the range of about 1 nm to about 20 nm, preferably less than about 10, 12, 14, 16, 17, or 20 nm. The nanoparticles can be of any shape including, rods, arrows, teardrops and tetrapods (see, e.g., Alivisatos et al., J. Am. Chem. Soc. 122:12700-12706 (2000)). Other suitable shapes include, e.g., square, round, elliptical, triangular, rectangular, rhombal and toroidal. The nanoparticles typically comprise a shell and a core. Typically the shell material will have a bandgap energy that is greater than the bandgap energy of the core material. In some embodiments, the shell material has an atomic spacing close to that of the core material. The term “monolayer” refers to each atomic layer of the shell material around the core. Each monolayer increases the diameter of the shell material, and increases the emission and total fluorescence of the core. The shell may further comprise a hydrophilic material (e.g., any compound with an affinity for aqueous materials such as H₂O). Nanoparticles include, e.g., semiconductor nanocrystals.

The terms “semiconductor nanocrystal,” “quantum dot,” and “qdot” are used herein to refer to luminescent semiconductor nanocrystals, i.e., nanoparticles comprising a core and a shell and capable of emitting electromagnetic radiation (i.e., a signal) upon excitation by an energy source (e.g., any source of electromagnetic radiation include a light source such as a microscope). Semiconductor nanocrystals are capable of absorbing and emitting radiation (i.e., luminescing) in response to a broad range of wavelengths, including the range from gamma radiation to microwave radiation. The semiconductor nanocrystals are also capable of emitting radiation within a narrow wavelength band of about 50, 40, 30, 20, or 10 nm or less.

The nanoparticles may be “conjugated” (i.e., linked) to the signal peptide directly or via one or more linking agents. “Linking agent” as used herein refers to any compound that forms a bond between the nanoparticle and the signal peptide and include e.g., a functional group, an affinity agent, or a stabilizing group. Suitable bonds include ionic interactions, covalent chemical bonds, physical forces such van der Waals or hydrophobic interactions, encapsulation, embedding, binding affinity, attraction or recognition, and various types of primary, secondary, tertiary linkages including but not limited to, peptide, ether, ester, acryl, aldehyde, ketone, acryloyl, thiol, carboxyl, hydroxyl, sulfhydryl and amine linkages or the like.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

As used herein, “signal peptide” refers to a peptide is capable of directing postranslation transport of a protein or other element (e.g., a signal peptide-nanoparticle conjugate) to specific locations within a cell such as, for example, the nucleus, peroxisomes, lysosomes, endosomes, CIIV, MIIC, mitochondria, endoplasmic reticulum, Golgi bodies, chloroplasts, and the cell membrane. Signal peptides can interact physically with biological compounds such as cells, proteins, nucleic acids, subcellular organelles and other subcellular components and can bind nonspecifically or sequence-specifically to nucleic acids (DNA RNA). Signal peptides also include small molecules that bind to the minor groove of DNA such as, for example the antigene peptide nucleic acids described in Janowski et al., Nature Chemical Biology 1, 210-215 (2005). In a preferred embodiment, a signal peptide is a polypeptide of about 5 to about 200, about 10 to about 150, about 15 to about 100, or about 20 to about 50 amino acids in length and directs transport of a signal peptide-nanoparticle conjugate to a cell nucleus.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region such as SEQ ID NOS:1-13), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Preferably, the identity exists over a region that is at least about 10, 15, 20, or 25 amino acids or nucleotides in length.

The term “substrate” herein refers to a solid support made of a material onto which nanoparticles (i.e., semiconductor nanocrystals) and/or cells can be deposited. Any material (e.g., glass, plastic, petri dish, cover slip or filter membrane) can be used for the substrate.

The term “culture surface” herein refers to a solid support suitable for deposition or incorporation of nanoparticles (i.e., semiconductor nanocrystals) and cells. The culture surface can be either the substrate itself or applied directly to the substrate.

The term “cell” refers to any cell from any animal or plant and includes cells from any tissue type. Animals from which cells can be derived include vertebrates such as mammals (e.g., rodents such as mice, rats, and guinea pigs; primates such as monkeys, chimpanzees, and humans; canines; felines; ovines; porcines; or bovine). Cell may also be unicellular organisms (e.g., bacteria or yeast).

“Cell mobility” as used herein refers to cellular movement (e.g., lamellipodial ruffling, crawling and gliding) and includes “motility,” “locomotion,” and “migration.” “Cell motility” or “locomotion” refers to spontaneous and/or non-directional movement of cells around or over a surface. “Cell migration” is used to describe the directional movement of cells from one tissue to another tissue, from tissue to blood stream to tissue, over and around a surface, or over and through a substrate (e.g., a filter). Cell migration includes chemotaxis. “Chemotaxis” refers to cell migration towards or away from a specific chemical stimulus, typically a concentration gradient (e.g., a gradient of salt, protein, sugar, or lipid).

Cell mobility can be detected by detecting the “phagokinetic track” generated by cell movement (i.e., both directional and non-directional movement). For example, a cell generates a phagokinetic track when it moves over a layer of “markers” (e.g., signal peptide-nanoparticle conjugates), and takes up the “markers,” leaving behind a region depleted of markers equal to the area the cell has traversed.

“Cellular uptake” as used herein refers to the action of cells bringing foreign material (e.g., signal peptide-nanoparticles) inside the cell. Cellular uptake includes, but is not limited to, mechanisms such as specific or non-specific engulfment, pinocytosis, endocytosis, and/or phagocytosis, or ingestion.

Since the detection of the signal peptide-semiconductor nanocrystal conjugate is based on the luminescence of the semiconductor nanocrystal portion of the conjugate, the signal peptide-semiconductor nanocrystal conjugates of the invention can be observed using any methods known in the art. Suitable methods include, e.g., fluorescence microscopy such as confocal and multi-photon microscopy. Semiconductor nanocrystals with many emission colors can be prepared and bio-conjugated to a range of targeting molecules, so that it is possible to monitor cell motility and migration, division and movement of the signal peptide-semiconductor nanocrystals within live cells.

By the term “about” it is meant, that it is contemplated that the size can be within ±5, 10, 15, 20, or 25 units or 5, 10, 15, 20, or 25% of the stated values.

By “diameter,” it is meant the length of a straight line passing through a figure (e.g., a signal peptide-nanoparticle conjugate), with the endpoints of the line at the widest position. Thus, for example, for a peptide-semiconductor nanocrystal conjugated to three signal peptides linked through streptavidin, the diameter should be measured at the widest point, likely from the end of one peptide to the end of another peptide.

“Biological sample” as used herein is a sample of biological tissue or fluid that is suspected of containing an analyte of interest. Samples include, for example, body fluids such as whole blood, serum, plasma, cerebrospinal fluid, urine, lymph fluids, and various external secretions of the respiratory, intestinal and genitourinary tracts such as tears, saliva, semen, milk, and the like; and other biological fluids such as cell culture suspensions, cell extracts, cell culture supernatants. Samples may also include tissues biopsies, e.g., from the lung, liver, brain, eye, tongue, colon, kidney, muscle, heart, breast, skin, pancreas, uterus, cervix, prostate, salivary gland, and the like. A sample may be suspended or dissolved in, e.g., buffers, extractants, solvents, and the like. A sample can be from any naturally occurring organism or a recombinant organism including, e.g., viruses, prokaryotes or eukaryotes, and mammals (e.g., rodents, felines, canines, and primates). The organism may be a nondiseased organism, an organism suspected of being diseased, or a diseased organism. A mammalian subject from whom a sample is taken may have, be suspected of having, or have a disease such as, for example, cancer, autoimmune disease, or cardiovascular disease, pulmonary disease, gastrointestinal disease, muscoskeletal disorders, central nervous system disorders, infectious disease (e.g., viral, fungal, or bacterial infection). The term biological sample also refers to research samples which have been deliberately created for the study of biological processes or discovery or screening of drug candidates. Such examples include, but are not limited to, aqueous samples that have been doped with bacteria, viruses, DNA, polypeptides, natural or recombinant proteins, metal ions, or drug candidates and their mixtures.

### III. Signal Peptide-Nanoparticle Conjugates

The signal peptide-nanoparticle conjugates described herein can enter any subcellular compartment and are typically less than about 10, 12, 14, 16, 18, or 20 nm in diameter. In a preferred embodiment, the conjugates are less than about 20 nm in diameter and comprise a signal peptide conjugated to a semiconductor nanocrystal. The semiconductor nanocrystal may optionally comprise a hydrophilic coating. In one embodiment, the signal peptide is attached to a linking agent, which recognizes and/or attaches to a second linking agent which is attached to the semiconductor nanocrystal (i.e., either directly or via a functional group). In another embodiment, the signal peptide is attached to the semiconductor nanocrystal by means of a bifunctional crosslinker as described herein.

The signal peptide and nanoparticle may be conjugated using any means known in the art (see, e.g., Chen and Gerion, Nano Letters 4(10:1827-1832 (2004); Gerion et al., J. Phys. Chem. 105(37):8861-8871 (2001); and Peng et al., J. Amer. Chem. Soc., 119(30):7019-7029 (1997)). Typically the nanoparticle (e.g. a semiconductor nanocrystal) is prepared using the methods described in, e.g., U.S. Pat. Nos. 5,751,018; 5,505,928; and 5,262,357, which describe methods for synthesizing and controlling the size of semiconductor nanocrystals comprising Group Ill-V semiconductors or Group II-VI semiconductors. In some embodiments, the nanoparticle is further treated to comprise a hydrophilic coating (e.g., a silica shell) as described in, e.g., Gerion et al., Chemistry of Materials, 14:2113-2119 (2002); Mattoussi et al., Physica Status Solidi B, 224(1):277-283 (2001); and Chan et al., Science, 281:2016-2018 (1998)). The coated or uncoated nanoparticle is further derivatized with a linking agent (e.g., functional groups, affinity agents, and linking groups) using methods known in the art and contacted with the signal peptide in an aqueous solution (e.g., a phosphate buffer) for an appropriate incubation time (e.g., 5 seconds to several hours, preferably from about 5 minutes to about 24 hours). Excess signal peptides are washed away. In some embodiments, the signal peptide is derivatized (e.g., to comprise an aldehyde group) prior to contact with the nanoparticle. Typically the ratio of signal peptide: nanoparticle is about 1:1, 2:1, 3:1, 4:1, 5:1, 6:1, 7:1, 8:1, 9:1, 10:1, 12:1, 15:1, 20:1, 30:1, 40:1, 50:1, 60:1, 70:1, 80:1, 90:1, or 100:1.

A. Signal Peptides

Any suitable signal peptide can be used in the signal peptide-nanoparticle conjugates of the invention. The peptide should be able to target (i.e., mediate entry and accumulation) of a signal peptide-nanoparticle to a subcellular compartment and/or organelle of interest. Signal peptides are typically about about 5 to about 200, about 10 to about 150, about 15 to about 100, or about 20 to about 50 amino acids in length. Suitable signal peptides include, e.g., nuclear localization signal peptides, peroxisome-targeting signal peptides, cell membrane-targeting signal peptides, mitochondrial-targeting signal peptides, and endoplasmic reticulum-targeting signal peptides, and trans-Golgi body-targeting signal peptides. Signal peptides may also target the signal peptide-nanoparticle conjugates to any cell surface receptor including e.g. epidermal growth factor receptors (EGFR), fibroblast growth factor receptors (FGFR), vascular endothelial cell growth factor receptor (VEGFR), integrins, chemokine receptors, platelet-derived growth factor receptor (PDGFR), tumor growth factor receptora, and tumor necrosis factor receptors (TNF).

Nuclear localization signal peptides typically comprise positively charged amino acids. Endoplasmic reticulum targeting signal peptides typically comprise about 5 to about 10 hydrophobic amino acids. Mitochondria targeting signal peptides are typically about 5 to about 10 amino acids in length and comprise a combination of hydrophobic amino acids and positively charged amino acids. Peroxisome targeting signal peptides include PTS1, a 3 amino acid peptide and PTS2, a 26-36 amino acid peptide. Golgi body targeting signal peptides are set forth in, e.g., Honda et al., J. Cell Biol 168, 1039-1051 (2005)); EGFR targgeting signal peptides are set forth in, e.g., Fan et al., J Biol Chem 279, 38143-38150 (2004); additional nuclear localization signal peptides are set forth in, e.g., Tkachenko et al., Bioconjug Chem 15, 482-490 (2004); integrin targeting signal peptides are set forth in, e.g., Kato et al., J Biol Chem 277, 28934-28941 (2002); and kinase targeting signal peptides are set forth in, e.g., van Hennik et al., J Biol Chem 278, 39166-39175 (2003). Examples of signal peptide sequences include but are not limited to the following sequences:

| | | |
|---|---|---|
| Target | Source | Sequence |

| | | |
|---|---|---|
| Nucleus | SV-40 large T | PPKKKRKVPPKKKRKV |
| | antigen | (SEQ ID NO: 1) |
| Nucleus | Tat protein of | YGRKKRRQRRR |
| | HIV | (SEQ ID NO: 5) |
| Endoplasmic | | KDELA KDELA KDELA KDEL |
| Reticulum | | (SEQ ID NO: 2) |
| Mitochondria | Cytochrome C | SVTTPLLLRGLTGSARRLPVPRAKIHSL |
| | oxidase | (SEQ ID NO: 3) |
| Peroxisome | | SKLA SKLA SKLA SKLA |
| | | (SEQ ID NO: 4) |
| Cell Membrane | | KLNPPDESGPCMSCKCVLS |
| | | (SEQ ID NO: 6) |
| Cell Membrane | GAP-43 | MLCCMRRTKQVEKNDEDQKI |
| | | (SEQ ID NO: 7) |
| Golgi Body | ARF-1 | (MXXE)₄ |
| | | (SEQ ID NO: 8) |
| EGFR | | EEEEYFELV |
| | | (SEQ ID NO: 9) |
| Nucleus | Adenovirus | CGGFSTSLRARKA |
| | | (SEQ ID NO: 10) |
| Integrin | | CKKKKKKGGRGDMFG |
| | | (SEQ ID NO: 11) |
| phosphatidylinositol | Rac1 | CPPPVICKKRKR |
| 4,5-phosphate | | (SEQ ID NO: 12) |
| kinase (PIP5K) | | |
| alpha(IIb)beta(3) | | KVGFFKR |
| integrin | | (SEQ ID NO: 13) |

Signal peptides can be chemically synthesized or recombinantly produced. In general, the nucleic acid sequences encoding signal peptides and related nucleic acid sequence homologues are cloned from cDNA and genomic DNA libraries or isolated using amplification techniques with oligonucleotide primers. Standard techniques are used for nucleic acid and peptide synthesis, cloning, DNA and RNA isolation, amplification and purification. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For example, signal peptides sequences are typically isolated from nucleic acid (genomic or cDNA) libraries by hybridizing with a nucleic acid probe, the sequence of which can be derived from a nucleic acid encoding SEQ ID NOS:1-13. Signal peptide RNA and genomic DNA can be isolated from any suitable mammal. Methods for making and screening cDNA libraries and genomic DNA libraries are well known (see, e.g., Gubler & Hoffman, Gene 25:263-269 (1983); Sambrook et al., supra; Ausubel et al., supra; Benton & Davis, Science 196:180-182 (1977); and Grunstein et al., PNAS USA, 72:3961-3965 (1975)). The sequence of the cloned signal peptides can be verified using, e.g., the chain termination method for sequencing double-stranded templates of Wallace et al., Gene 16:21-26 (1981).

To obtain high level expression of a cloned cDNAs encoding signal peptides, one typically subclones a signal peptide sequence (i.e., a sequence encoding SEQ ID NOS:1-7) into an expression vector that is subsequently transfected into a mammalian cells. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding a signal peptide (e.g., a sequence encoding SEQ ID NOS:1-7). Eukaryotic expression systems for mammalian cells are well known in the art and described, e.g., in Sambrook et al. and Ausubel et al. and are also commercially available. Preferably the mammalian cells are cells that do not endogenously express the signal peptide.

The particular expression vector used to transport the genetic information into the cell is not particularly critical. Any of the conventional vectors used for expression in eukaryotic cells may be used. Preferred vectors include vectors that comprise a strong promoter/enhancer region such as, e.g., pCI-neo, or vectors that comprise multiple cloning sites followed by an internal ribosome entry site (IRES) and/or a sequence encoding a marker protein such as, e.g., pIRES2-EGFP. The vectors also typically comprise a gene encoding antibiotic resistance to permit selection of cells have been transformed with the vector. The particular antibiotic resistance gene chosen is not critical, any of the many resistance genes known in the art are suitable. Preferably the antibiotic resistance gene confers resistance to neomycin.

Standard transfection methods are used to produce cell lines that express large quantities of a signal peptide, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss and Curtiss, METHODS ENZYMOLOGY 101:347-362 (Wu et al., eds, 1983).

For example, any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the signal peptide.

After the expression vector is introduced into the cells, the transfected cells are cultured under conditions favoring expression of the signal peptide. Once expressed, signal peptides may be purified to substantial purity by standard techniques known in the art, including, for example, size differential filtration, solubility fractionation, selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant signal peptides are being purified. For example, proteins having established molecular adhesion properties can be reversible fused to signal peptides. With the appropriate ligand, the signal peptides can be selectively adsorbed to a purification column and then freed from the column in a relatively pure form. The fused protein is then removed by enzymatic activity. Finally the signal peptides could be purified using immunoaffinity columns.

Signal peptides can also be derivatized during chemical synthesis or following purification. In some embodiments, the signal peptides are derivatized to comprise a reactive group (e.g., an aldehyde group) that will interact with the bifunctional linkers described in detail below.

B. Nanoparticles

The nanoparticle portion of the conjugates described herein typically comprise a core and a shell. The core and the shell may comprise the same material or different materials. The shell may further comprise a hydrophilic coating or another group that facilitates conjugation of a signal peptide to a nanoparticle (i.e., via a linking agent). In some embodiments, the semiconductor nanocrystals comprise a core upon which a hydrophilic coating has been deposited.

The core and the shell may comprise, e.g., an inorganic semiconductive material, a mixture or solid solution of inorganic semiconductive materials, or an organic semiconductive material. Suitable materials for the core and/or shell include, but are not limited to semiconductor materials, carbon, metals, and metal oxides. In a preferred embodiment, the nanoparticles comprise a semiconductor nanocrystal. In a particularly preferred embodiment, the semiconductor nanocrystals comprise a CdSe core and a ZnS shell which further comprises a SiO₂ hydrophilic coating.

The core typically has a diameter of about 1, 2, 3, 4, 5, 6, 7, or 8 nm. The shell typically has thickness of about 1, 2, 3, 4, 5, 6, 7, or 8 nm and a diameter of about 1 to about 10, 2 to about 9, or about 3 to about 8 nm. In a preferred embodiment, the core is about 2 to about 3 nm in diameter and the shell is about 1 to about 2 nm in thickness.

Suitable semiconductor materials for the core and/or shell include, but are not limited to, elements of Groups II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like), and alloys or mixtures thereof. Suitable metals and metal oxides for the core and/or shell include, but are not limited to, Au, Ag, Co, Ni, Fe₂O₃, TiO₂, and the like. Suitable carbon nanoparticles include, but are not limited to, carbon nanspheres, carbon nano-onions, and fullerene.

Semiconductor nanocrystals can be made using any method known in the art. For example, methods for synthesizing semiconductor nanocrystals comprising Group III-V semiconductors or Group II-VI semiconductors are set forth in, e.g., U.S. Pat. Nos. 5,751,018; 5,505,928; and 5,262,357. The size of the semiconductor nanocrystals can be controlled during formation using crystal growth terminators U.S. Pat. Nos. 5,751,018; 5,505,928; and 5,262,357. Methods for making semiconductor nanocrystals are also set forth in Gerion et al., J. Phys. Chem. 105(37):8861-8871 (2001) and Peng et al., J. Amer. Chem. Soc., 119(30):7019-7029 (1997).

The semiconductor nanocrystals may further comprise a hydrophilic coating (e.g., a coating of hydrophilic materials or stabilizing groups) to enhance the solubility of the nanocrystals in an aqueous solution. Typically the hydrophilic coating is about 0.1, 0.25, 0.5, 0.75, 1, 2, 3, 4, 5, 6, 7, 8, 9, or 10 nm thick. Suitable hydrophilic materials include, e.g., SiO, SiO₂, polyethylene glycol, ether, mecapto acid and hydrocarbonic acid, and dihydroxylipoic acid (DHLA). Suitable stabilizing groups include, e.g. positively or negatively charged groups or groups that facilitate steric repulsion. In a preferred embodiment, the hydrophilic coating is a silica shell (e.g., comprising SiO₂). Methods of silanizing semiconductor nanocrystals are well known in the art and are described in, e.g., Gerion et al., Chemistry of Materials, 14:2113-2119 (2002). Other methods for generating water-soluble semiconductor nanocrystals are described in, e.g., Mattoussi et al., Physica Status Solidi B, 224(1):277-283 (2001) and Chan et al., Science, 281:2016-2018 (1998).

In a particularly preferred embodiment, the hydrophilic coating comprises a silica shell having a thickness of about 0.5 to about 5, about 1 to about 4, or about 2 to about 3 nm. Preferably the silica shell is amorphous and porous. Silica shells can be deposited on the core or the shell of the semiconductor nanocrystal using the methods described in, e.g., Alivisatos et al., Science, 281:2013-2016 (1998) and Gerion, et al., J. Phys. Chem. 105(37):8861-8871 (2001). In a particularly preferred embodiment, the semiconductor nanocrystals have core/shell configuration of CdSe/ZnS/SiO₂ wherein the layers are about 25/5/50 Å respectively from the center of the core.

The wavelength band emitted from the semiconductor nanocrystal is related to the physical properties (e.g., size, shape, and material), of the semiconductor nanocrystal. More particularly, the wavelength band emitted by the semiconductor nanocrystals may be affected by (1) the size of the core; (2) the size of the core and the size of the shell; (3)the composition of the core and shell. For example, a semiconductor nanocrystal comprised of a 3 nm core of CdSe and a 2 nm thick shell of CdS will emit a narrow wavelength band of light with a peak intensity wavelength of 600 nm. In contrast, a semiconductor nanocrystal comprised of a 3 nm core of CdSe and a 2 nm thick shell of ZnS will emit a narrow wavelength band of light with a peak intensity wavelength of 560 nm. As another example, when a 1-10 monolayer thick shell of CdS is epitaxially grown over a core of CdSe, there is a dramatic increase in the room temperature photoluminescence quantum yield.

Thus, one of skill in the art will appreciate that any of the physical properties of the semiconductor nanocrystals can be modified to control the wavelength band of the semiconductor nanocrystal and the corresponding signal peptide-semiconductor nanocrystal conjugate. For example, the composition of the semiconductor nanocrystal core or shells can be varied and the number of shells around the core of the semiconductor nanocrystal can be varied. In addition, semiconductor nanocrystals comprising different core materials, but the same shell material can be synthesized. Semiconductor nanocrystals comprising the same core material, but the different shell materials can also be synthesized.

C. Linking Agents

The signal peptide-nanoparticle conjugates are typically attached via a linking agent. The signal peptide and nanoparticle can be conjugated via a single linking agent or multiple linking agents. For example, the signal peptide and nanoparticle may be conjugated via a single multifunctional (e.g., bi-, tri-, or tetra-) linking agent or a pair of complementary linking agents. In some embodiments, the signal peptide and the nanoparticle are conjugated via two, three, or more linking agents. Suitable linking agents include, e.g., functional groups, affinity agents, stabilizing groups, and combinations thereof.

### 1. Functional Groups

Functional groups include monofunctional linkers comprising a reactive group as well as multifunctional crosslinkers comprising two or more reactive groups capable of forming a bond with two or more different functional targets (e.g., peptides, proteins, macromolecules, semiconductor nanocrystals, or substrate). In some preferred embodiments, the multifunctional crosslinkers are heterobifunctional crosslinkers comprising two different reactive groups.

Suitable reactive groups include, e.g., thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, e.g., haloacetyl and alkyl halide derivates, maleimides, aziridines, acryloyl derivatives, arylating agents, and thiol-disulfides exchange reagents. Carboxylate reactive groups include, e.g., diazoalkanes and diazoacetyl compounds, such as carbonyldiimidazoles and carbodiimides. Hydroxyl reactive groups include, e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, e.g., hydrazine derivatives for schiff base formation or reduction amination. Active hydrogen reactive groups include, e.g., diazonium derivatives for mannich condensation and iodination reactions. Photoreactive groups include, e.g., aryl azides and halogenated aryl azides, benzophenones, diazo compounds, and diazirine derivatives.

Other suitable reactive groups and classes of reactions useful in practicing the present invention are generally those that are well known in the art of bioconjugate chemistry. Currently favored classes of reactions available with reactive chelates are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

In one preferred embodiment, the functional group is a heterobifunctional crosslinker comprising two different reactive groups that form a heterocyclic ring that can interact with a signal peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfulydryl and photoreactive groups; carbonyl and photoreactive groups; carboxylate and photoreactive groups; and arginine and photoreactive groups.

### 2. Affinity Agents

In some embodiments, an affinity agent (e.g., agents that specifically binds to a ligand) is the linking agent. In these embodiments, a first linking agent is bound to the semiconductor nanocrystal and a second linking agent is bound to the signal peptide. Affinity agents include receptor-ligand pairs, antibody-antigen pairs and other binding partners such as streptavidin/avidin and biotin. In a preferred embodiment, the first linking agent is streptavidin or avidin and the second linking agent is biotin. When the streptavidin or avidin is bound to the seminconductor nanocrystal and a biotinylated signal peptide is conjugated to the semiconductor nanocrystal via streptavidin/avidin-biotin linkage. In some embodiments, other biotinylated peptides, proteins, antibodies, dyes, probes and other small molecules are attached to the streptavidin or avidin, and thus the semiconductor nanocrystal.

In general, any affinity agent useful in the prior art, in combination with a known ligand to provide specific recognition of a detectable substance will find utility in the formation of the signal peptide-semiconductor nanocrystal conjugates of the invention. Examples of suitable affinity agents, include but are not limited to, polysaccharides, lectins, selectins, nucleic acids (both monomeric and oligomeric), proteins, enzymes, lipids, monoclonal and polyclonal antibodies or fragments thereof (e.g., Fab, Fv, and scFv), and small molecules such as sugars, peptides, aptamers, drugs, and ligands.

In some embodiments, organic molecules are linked to the semiconductor nanocrystal may also influence cellular uptake or localization as described in, e.g., U.S. Pat. Nos. 5,990,479; 6,207,392 and 6,699,723.

### 3. Stabilizing Groups

In some embodiments, the linking agent is a stabilizing group (i.e., a group that stabilizes semiconductor nanocrystals in aqueous solutions such as water) that can be attached to the semiconductor nanocrystals. Suitable stabilizing groups include, for example, phosphonate (negatively charged), carboxyl (negatively charged), polyethylene glycol (PEG which is neutral and provides steric repulsion), and ammonium (positively charged).

Methods of making negatively and positively charged semiconductor nanocrystals are described by Gerion et al., Journal of Physical Chemistry, 105(37):8861-8871 (2001).

### IV. Assays Using Signal Peptide-Nanoparticle Conjugates

The signal peptide-nanoparticle conjugates described herein can be used in a variety of assays that require labeled cells or subcellular compartments. Subcellular compartments or structures that can be targeted include, but not limited to, the cell membrane, nucleus, peroxisome, mitochondria, smooth and rough endoplasmic reticulum, lysosomes, chloroplasts, Golgi bodies, and ribosomes.

The signal peptide-nanoparticle conjugates can be introduced into the cell (i.e., transfected) using any methods known in the art including, e.g., electroporation, lipofectamine transfection, or by spontaneous cellular uptake. Typically, the signal peptide-nanoparticle conjugates are contacted with the cells in an aqueous solution (e.g. buffer or culture medium) in a ratio of about 10, 20, 50, 100, 200, 250, 500, 750, or 1000 pmol nanoparticles to 1×10⁵ cells in a volume of about 0.5, 1, 2, 3, 4, 5, 6, 7, 8, 9, or 10 ml. The signal peptides and nanoparticles are incubated for an appropriate time (e.g., 1, 2, 5, 10, 20, 30, 40, 50, 60 or more minutes) prior to electroporation or lipofectamine transfection. Following transfection, the cells may be maintained in an aqueous solution or transferred to a solid support (e.g., a glass slide) for detection of the signal peptide-nanoparticle conjugates.

To detect the location of the signal peptide-nanoparticle conjugates, a light source excites the semiconductor nanocrystals and images are collected and recorded by an imaging system. In one embodiment, an upright epi-fluorescent microscope is used to collect fluorescence and phase contrast (PC) images. The PC images shows where the cells are, the fluorescence image shows the location of the where the signal peptide-semiconductor nanocrystal conjugates. By overlaying the images, the location of the signal peptide-semiconductor nanocrystal conjugates in the cells can be visualized.

Methods and devices for cellular imaging using semiconductor nanocrystals are also described in, e.g., U.S. Patent Publication No. 20030113709.

A. Cells

Cells which can be used in the assays described herein include eukaryotic and prokaryotic cells. Suitable cells include, e.g., epithelial cells, fibroblasts, and macrophages. Other suitable cell types include, e.g., cells that exhibit migratory behavior (e.g., fibroblasts) and cells that exhibit chemotactic behavior (e.g. macrophages). Suitable cells also include, microorganisms such as, for example, bacteria (e.g., E. coli, Listeria, and Salmonella), yeast (e.g., Saccharomyces), algae (e.g., Chlamydomonas).

This invention relies upon routine techniques in the field of cell culture. Suitable cell culture methods and conditions can be determined by those of skill in the art using known methodology (see, e.g., Freshney et al., CULTURE OF ANIMAL CELLS (3rd ed. 1994)). In general, the cell culture environment includes consideration of such factors as the substrate for cell growth, cell density and cell contract, the gas phase, the medium, and temperature.

Incubation is generally performed under conditions known to be optimal for cell growth. Such conditions may include for example a temperature of approximately 37° C. and a humidified atmosphere containing approximately 5% CO₂. The duration of the incubation can vary widely, depending on the desired results. Proliferation is conveniently determined using ³H thymidine incorporation or BrdU labeling.

Plastic dishes, flasks, roller bottles, or microcarriers in suspension may be used to culture cells according to the methods of the present invention. Suitable culture vessels include, for example, multi-well plates, petri dishes, tissue culture tubes, flasks, roller bottles, and the like.

Cells are grown at optimal densities that are determined empirically based on the cell type. Cells are passaged when the cell density is above optimal.

Cultured cells are normally grown in an incubator that provides a suitable temperature, e.g., the body temperature of the animal from which is the cells were obtained, accounting for regional variations in temperature. Generally, 37° C. is the preferred temperature for cell culture. Most incubators are humidified to approximately atmospheric conditions.

Important constituents of the gas phase are oxygen and carbon dioxide. Typically, atmospheric oxygen tensions are used for cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media include DME, RPMI 1640, DMEM, Iscove's complete media, or McCoy's Medium (see, e.g., GibcoBRL/Life Technologies Catalogue and Reference Guide; Sigma Catalogue). Typically, RPMI 1640 is used in the methods of the invention. Defined cell culture media are often supplemented with 5-20% serum, typically heat inactivated serum, e.g., human, horse, calf, and fetal bovine serum. Typically, 10% fetal bovine serum is used in the methods of the invention. The culture medium is usually buffered to maintain the cells at a pH preferably from 7.2-7.4. Other supplements to the media include, e.g., antibiotics, amino acids, sugars, growth factors, yeast extracts, soy extracts, and rice extracts. Typically, the media is supplemented with glucose at about 5.5-16.7 mM. In some embodiments, the glucose is present at about 11 mM.

One of skill in the art will appreciate that any of these conditions may be optimized for the particular cell type (e.g., mammalian cells, yeast cells, or bacterial cells) being cultured.

B. Transfection

The cells may be transfected with the signal peptide-nanoparticle conjugates using electroporation using any convenient method (see, e.g., Sambrook et al, supra) discussed herein.

In a preferred embodiment, the cells are transfected with the signal peptide-nanoparticle conjugates using electroporation. Electroporation conditions include: electric field strength, pulse duration, pulse number, and pulse frequency. One of skill in the art will understand that appropriate values for each of these factors, i.e., values that enhance transfection efficiency, can be determined by standard means known in the art, i.e., without undue experimentation. (See, e.g., Canatella and Prausnitz, Gene Therapy 8:1464 (2001)). The electrode may emit an electric field strength from about 1 to about 1000 V/cm, from about 25 to about 750 V/cm, from about 50 to about 500 V/cm, form about 60 to about 300 V/CM or from about 75 to about 250 V/cm. The pulse length may be from about 1 to about 60 ms, from about 2 to about 50 ms, from about 4 to about 40 ms, from about 5 to about 30 ms, or from about 7 to about 25 ms. For example, a suitable electric field strength is typically from about 100 V/cm to about 200 V/cm and a suitable electrical pulse length is typically from about 10 ms to about 20 ms. A suitable number of pulses is typically from about 1 to about 30 pulses, from about 2 to about 20 pulses, from about 4 to about 15 pulses, from about 5 to about 12 pulses, preferably from about 5 pulses to about 6 pulses.

Suitable signal generators for electroporation are commercially available and include, for example, an Electro Cell Manipulator Model ECM 600 (Genetronics, Inc., San Diego, Calif.), an Electro Cell Manipulator Model ECM 830 (BTX, San Diego, Calif.), an ElectroSquarePorator T820 (Genetronics, Inc., San Diego, Calif.), a PA-2000 (Cyto Pulse Sciences, Inc., Columbia, Md.) or a PA-4000, (Cyto Pulse Sciences, Inc., Columbia, Md.). These signal generators and methods of using them are described in U.S. Pat. Nos.: 6,314,316, 6,241,701, 6,233,482, 6,135,990, 5,993,434, and 5,704,908.

C. Cell Movement

In another aspect of the invention, the signal peptide-nanoparticle (e.g., semiconductor nanocrystal) conjugates may be used to label cells to study their motility and migration (e.g., in response to a chemotactic agent). An apparatus and methods for assaying cell migration in response to a chemotactive agent are also described in, e.g., U.S. Pat. No. 6,238,874. The apparatus comprises a chamber with two regions. A concentration gradient of the chemotactic agent (e.g., salt, sugar, lipid, or protein)which increases progressively (i.e., higher or lower) from the first region to the second region (i.e., the interrogation region) is established. Cells labeled with signal peptide-nanoparticle conjugates are placed in the chamber and are detected by comparing their distribution in the interrogation region at two or more time intervals. In some embodiments, the positional information for each cell or a population of cells is detected over a period of time by tracking the fluorescence emitted by the nanoparticle (e.g., a semiconductor nanocrystal) portion of the conjugate.

In some embodiments, a homogenous layer of signal peptide-nanoparticle conjugates are first deposited on a substrate (e.g., a solid support such as a glass slide). The substrate may comprise a gradient of a chemotactic agent. The cells are then contacted with the substrate under conditions such that the cells take up the conjugates. The cells will then exhibit motility or migrate in response to the chemotactic agent, thereby leaving behind a fluorescence-free trail (i.e., phagokinetic track). By subsequently determining the ratio of cell area to fluorescence-free track area, it is possible to track cell movement and differentiate between, e.g., invasive and noninvasive cancer cells and for studying cell signaling events involved in migration. Such assays are described in, e.g., Gu et al., Sci STKE 2005 Jun 28;2005(290) (2005)).

In some embodiments, the signal peptide-nanoparticle conjugates comprise a plurality of semiconductor nanocrystals, each of which has a different emission wavelength band. In such embodiments, it is possible to monitor motility (e.g., of a potentially metastatic cell) and migration of a number of different cell populations (e.g., in response to the chemotactic agent), each of which is labeled with a signal peptide-nanoparticle conjugate which has a different emission wavelength band.

In some embodiments, the signal peptide-nanoparticle conjugates comprise a plurality of signal peptides, each of which targets the conjugate to a different subcellular compartment. In such embodiments, it is possible to monitor the subcellular trafficking mechanisms related to the signal peptides.

In some embodiments, the signal peptide-nanoparticle conjugates comprise a plurality of semiconductor nanocrystals, each of which has a different emission wavelength band and a plurality of signal peptides, each of which targets the conjugate to a different subcellular compartment. In such embodiments, it is possible to simultaneously monitor the movement of the conjugates and the motility and migration of the cells.

D. Continuous Imaging of Live Cells

In some embodiments, the signal peptide-nanoparticle conjugates of the invention can be used to continuously image and track the motility of the signal peptide-semiconductor nanocrystals in live cells for more than 1, 2, 4, 6, 8, 12, 24 hours, 2, 4, 6, 8, 10, 12 weeks, 1, 2, 4, 6 weeks, and intervals thereof. Because daughter cells retain the signal peptide-semiconductor nanocrystals taken up or located in the cellular compartments following mitosis, the signal peptide-semiconductor nanocrystals can also conveniently be used to generate a population of labeled cells to study and track cell lineage, growth and division.

E. Detection of Signal Peptide-Nanoparticle Conjugates

Detection of the signal peptide-nanoparticle (i.e., semiconductor nanocrystal) conjugates can be by any means known in the art. Optical excitation of the semiconductor nanocrystal is preferable, however, electromagnetic radiation of wavelength ranging from x-ray to ultraviolet to visible to infrared waves may be used to excite the semiconductor nanocrystals. In general, any light source with an emission spectrum with a wavelength shorter than the wavelength of emission of the semiconductor nanocrystals can be used. Examples of effective light sources include, but are not limited to, a high-intensity light source such as a mercury lamp or xenon lamp, a laser, halogen lamp, LED, or hand-held UV lamps. Although x-rays and electronic beams may be used, there may be problems using these in the present invention due to absorption of the beams by other elements of the invention such as petri dishes or water.

The present semiconductor nanocrystals are illuminated by a light source, preferably by a laser, and more preferably by a krypton-argon laser or Ti-Sapphire laser. The light source excites the semiconductor nanocrystals and images are collected and recorded by an imaging system. Many light sources are possible and appropriate for use in the invention. For epi-fluorescence, one can use a mercury lamp or xenon lamp and record the image using a 35 mm camera, a digital camera, or a CCD camera. Many lasers can be used, including argon, krypton-argon, helium-neon, and Ti-sapphire lasers typically used with confocal and multi-photon microscopy

Illumination of the semiconductor nanocrystals enables a user to measure the amount of semiconductor nanocrystal conjugates taken up by at least one cell. This can be done by methods that include, but are not limited to the following.

In one embodiment, each cell can be viewed and the vesicles containing the engulfed semiconductor nanocrystal conjugates can be imaged.

In some embodiments, a dye may be used to label cell features in order to improve the visibility of the engulfed semiconductor nanocrystal conjugates. Suitable dyes include, e.g., FM4-64 and FM 5-95 (T-23360) from Molecular Probes, Inc., both of which stain with red fluorescence (excitation/emission maxima ˜515/640 nm).

Imaging of cells that have taken up semiconductor nanocrystal conjugates can be carried out by various means which include, but are not limited to, confocal fluorescence microscopy and multi-photon microscopy etc. Images, for example, can be collected with a confocal microscope using fluorescence detectors to examine the semiconductor nanocrystal conjugates and DIC (Differential Interference Contrast) to visualize the cells. Two-photon excitation at appropriate wavelengths with a Ti-sapphire laser or a krypton-argon laser can be used to excite the semiconductor nanocrystals so that optical sections of the cells can obtained using confocal microscopy with a 40×1.3 NA lens.

F. Diagnostic and/or Therapeutic Methods

It is further contemplated that the signal peptide-semiconductor nanocrystal conjugates can be used to tag molecules for use in diagnostic and/or therapeutic purposes. Using the signal peptide-semiconductor nanocrystal conjugates, the tracking of molecules throughout a cell can be easily facilitated. Such molecules include but are not limited to, peptides, proteins, oligonucleotides, drug delivery vehicles, toxins, viruses, therapeutics, fluorescent dyes, radiolabeled dyes, and other dyes and probes, polysaccharides, lectins, selecting, nucleic acids (both monomeric and oligomeric), dendrimers, enzymes, lipids, antibodies, and small molecules such as sugars, peptides, aptamers, drugs, and ligands. The molecules may be labeled to facilitate detection.

For example, in some embodiments, the signal peptide-nanoparticle conjugates disclosed herein can be further conjugated to antibodies that specifically bind to nuclear proteins used to image targeted nuclear proteins and biological processes in the nucleus.

In some embodiments, the signal peptide-seminconductor nanocrystal conjugates are used in multiplex assays. Because of the broad bandwidth at which the semiconductor nanocrystals can be excited to emit a luminescent signal, a common excitation source can be used for the simultaneous excitation of several semiconductor nanocrystals, i.e., several semiconductor nanocrystals which give off radiation at different frequencies, thus permitting simultaneous excitation and detection of the presence of several semiconductor nanocrystals indicating, for example, the presence of several detectable substances in the material (e.g., a biological sample or cell population) being examined.

In some embodiments, the signal peptide-semiconductor nanocrystal conjugates of the invention can be used to compare the effects of a panel of test compounds on a particular cell type. By using conjugates comprising semiconductor nanocrystals that luminesce at different wavelength, the effect of multiple test compounds can be measured simultaneously.

In some embodiments, the signal peptide-semiconductor nanocrystal conjugates can be used to determine how different biological molecules (i.e., biomolecules) affect the cellular uptake of semiconductor nanocrystals. For example, by attaching different types of biomolecules (e.g., peptides, nucleic acids, lipids, polysaccharides, and the like) to semiconductor nanocrystals, the effect of the biomolecules on the rate, quantity, and other parameters of nanocrystal uptake by cells can be determined.

The number of colors of semiconductor nanocrystals that can be resolved in parallel can be calculated on the rule of Full Width Half Maximum (FWHM). For example, based on the rule of FWHM and a wavelength range of approximately 400 to 800 nanometers, approximately 20 different colors of semiconductor nanocrystals could be resolved in parallel.

### V. Kits

The present invention also provides kits for imaging cells or subcellular compartments. Such kits typically comprise two or more components necessary for such imaging techniques. Components may be compounds, reagents, containers and/or equipment. In some embodiments, one container within a kit may contain a control signal peptide-nanoparticle (e.g., semiconductor nanocrystal) conjugate comprising a scrambled signal peptide sequence and other containers within the kit may contain a signal peptide-nanoparticle (e.g., semiconductor nanocrystal) comprising a signal peptide that targets the semiconductor nanocrystal to the nucleus, transmembrane protein, endoplasmic reticulum, peroxisome, or Golgi body of a cell. In addition, the kits comprise instructions for use, i.e., instructions for transfecting cells with the nanoparticle-signal peptide conjugates and using the conjugates in the imaging assays and cell motility assays described herein. The kits may further comprise any of the reaction components or buffers described herein.

### EXAMPLES

The following examples are provided to illustrate, but not to limit the claimed invention.

### Example 1

### Making a Peptide-Semiconductor Nanocrystal using STV and SV40 Nuclear Localization Signal Peptide

A compact (˜10-15 nm) complex with the SV40 nuclear localization signal peptide was attached to a semiconductor nanocrystal as described herein. Peptide-semiconductor nanocrystal conjugates with a random peptide sequence and silanized semiconductor nanocrystals (emission ˜550 nm, fwhm ˜35 nm, quantum yield ˜25%) are prepared according to methods as described in Gerion et al., J. Phys. Chem. 105:8861-8871 (2001). The average size of the semiconductor nanocrystals used was about 8-10 nm.

The peptide sequences were bound to the silanized semiconductor nanocrystals through a streptavidin-biotin bridge. First, streptavidin-maleimide (STV) (Sigma) is covalently linked to the thiols of silanized dots. We used a ratio of STV: semiconductor nanocrystals of 2:1 to 4:1 in a 10 mM phosphate buffer, pH˜7 with 10% of formamide, and overnight reaction. Excess unbound STV was removed by several runs of centrifugation in a Centricon 100 device.

The linking of STV to the semiconductor nanocrystals is probed by an assay where non-fluorescent biotinylated microbeads are incubated with STV-semiconductor nanocrystals solutions. The binding of two colors of STV-semiconductor nanocrystals to biotinylated microbeads produces fluorescent beads. Microbeads exposed to red STV-semiconductor nanocrystals fluoresce in red and those exposed to green STV-semiconductor nanocrystals fluoresce in green. Control experiments, where STV is missing on the semiconductor nanocrystals or where it is blocked by an excess of biotin, show no fluorescence.

The STV-semiconductor nanocrystal conjugates are then linked to biotinylated NLS sequences [Nₜₑᵣₘᵢₙₐₗ-(Pro Pro Lys Lys Lys Arg Lys Val)₂-Cₜₑᵣₘᵢₙₐₗ Biotin] or to a biotinylated random peptide sequence [biotin Nₜₑᵣₘᵢₙₐₗ-Glu Pro Pro Leu Ser Gln Glu Ala Phe Ala Asp Leu Leu Lys Lys Lys-Cₜₑᵣₘᵢₙₐₗ], hereafter called “RP-semiconductor nanocrystals”. This conjugation step is performed in 10 mM sodium phosphate buffer, 150 mM NaCl, pH˜7.3 at room temperature. For labeling experiments a ratio of peptide:semiconductor nanocrystals of 5:1 to 10:1 with 20 min of reaction time was used. The incorporation of biotinylated peptides onto the STV-semiconductor nanocrystal conjugates under these conditions is best probed by gel electrophoresis, where the ratio of peptide:semiconductor nanocrystals spans from 1:1 to 50:1. The ratio of NLS:semiconductor nanocrystals is 0, 1, 3, 5, 10, 15, 20, 50, 50, 50, 0, 0.

Negatively charged STV-semiconductor nanocrystal conjugates exhibit a reduced mobility when incubated with the biotinylated NLS peptide, and the overall mobility decreases as the concentration of NLS increases. When free biotin is added to the reaction. 15 minutes after the NLS and is kept reacting for 15 additional minutes prior to running the gel, the free biotin seems capable of displacing the NLS from the STV-semiconductor nanocrystals. In some experiments, free biotin was added prior to the NLS addition and saturated the STV binding site. The presence of a 50-fold excess of NLS does not affect markedly the mobility of STV-semiconductor nanocrystals whose binding sites are blocked by biotin. This suggests that at concentrations used for transfection (peptide:qdot=5:1), the non-specific binding of the peptide is negligible.

At a ratio of 50 NLS per semiconductor nanocrystal, the NLS-semiconductor nanocrystal conjugates do not move from the well, yet such complexes do not show sign of bulk aggregation for weeks. Also, the post-addition of free biotin to the NLS-semiconductor nanocrystal conjugates that do not migrate restores a partial mobility to the semiconductor nanocrystals. Given the timeframe for such an experiment (30 min of electrophoresis), it is very unlikely that such a pattern represents diffusion of the particles in the gel. Rather, free biotin is likely, able to displace some biotinylated-NLS, thus reducing the number of positively charged peptides bound to the semiconductor nanocrystals and increasing the NLS-semiconductor nanocrystal mobility. This implies that the NLS peptide does not bind nonspecifically to STV-semiconductor nanocrystals. An additional indication in that direction is the fact that if free biotin saturates the STV binding sites of STV-semiconductor nanocrystals, the addition of a 50-fold excess of NLS has a very slight effect on the semiconductor nanocrystal mobility.

### Example 2

### Entry into Cellular Nuclei by Peptide-Semiconductor Nanocrystals

While the attachment of the peptide to semiconductor nanocrystals can be studied by test-tube experiments, their activity cannot. The only way to probe the biological activity of NLS-semiconductor nanocrystal conjugates is to check if the complexes are indeed able to target the cell nucleus. In this regard, the first step is to find an efficient way of introducing the signal peptide-semiconductor nanocrystal conjugates inside a large number of living cells. Two methods were investigated: electroporation and lipofectamine transfection. However, this example will focus only on electroporation. The electroporation of 2 types of semiconductor nanocrystals was investigated: “NLS-semiconductor nanocrystals”, i.e., semiconductor nanocrystals conjugated to the SV-40 large T antigen nuclear localization sequence; and “RP-semiconductor nanocrystals”, i.e., semiconductor nanocrystals conjugated to a random peptide, which were both made as described in Example 1.

The cell model system consisted of human HeLa cells grown as monolayer cultures in a humidified, 5% CO₂ atmosphere with α-minimal essential medium supplemented with 10% heat-inactivated fetal calf serum. The cells were trypsinized, resuspended in PBS, counted using a Coulter Counter, and diluted at 1×10⁵ cells/ml. Semiconductor nanocrystals were mixed with the cells at a ratio of 10 pmol of semiconductor nanocrystals/1×10⁵ cells, for a final concentration of 10 nM of semiconductor nanocrystals and 1 ml total volume. Electroporation was carried out in a 4 mm electroporation cuvette (Bio-Rad, CA) using 300V, 250 μF, and a pulse time of 5-6 msec with a Gene-Pulser II (Bio-Rad, CA). The cells were then seeded on the slide chamber (NUNC). Attachment of the cells to the slide surface was achieved either with centrifugation in a Beckman benchtop centrifuge at a speed of 1000 rpm for 2 minutes or by natural sedimentation for at least 2 hours.

HeLa cells transfected with NLS-semiconductor nanocrystals and cells transfected with semiconductor nanocrystals conjugated to a random peptide sequence were viewed under a 100× oil immersion objective using fluorescence imaging with an upright Olympus microscope BX51. The illumination source was a Hg lamp, and images were recorded with a Peltier-cooled CCD camera. Integration time varied from 200 msec to 700 msec per frame. For the tracking of the movement of qdots, the cells were illuminated continuously for periods of up to 1 hour, and images were taken every 15 seconds and subsequently processed.). All images are taken within 24 hrs after transfection. The NLS-semiconductor nanocrystal conjugates were observed either in the cell nucleus or in the perinuclear region. The percentage of cells with NLS-semiconductor nanocrystals localized in the nucleus is ˜15%, while in ˜85% of the cases, NLS-semiconductor nanocrystals accumulate preferentially in the perinuclear region. In contrast, semiconductor nanocrystals conjugated to a random peptide localize randomly in the cells and these conjugates are not seen inside the nucleus within the 24 hr time frame. The qualitative and distinct localization of the NLS-semiconductor nanocrystals and RP-semiconductor nanocrystals seems to preclude a passive mechanism, such as free diffusion, for the semiconductor nanocrystals entry into the nucleus, as it was previously observed as a result of disruption of nuclear membrane during multiple cell division cycles (Dubertret et al., Science 298:1759-1762 (2002)). Remarkably, when the NLS-semiconductor nanocrystal conjugates are in the nucleus, finer structures within the nucleus are revealed, such as the nucleoli.

### Example

### Effect of Size of Semiconductor Nanocrystal-Peptide conjugates on Cell Nucleus Entry

An intriguing question remains as to why certain NLS-semiconductor nanocrystals can all enter the nucleus of some HeLa cells, while NLS-semiconductor nanocrystals from the same transfection all get stuck in the perinuclear region of some other cells. Since the same NLS-semiconductor nanocrystal conjugates are used, the partial aggregation of the semiconductor nanocrystals or their incorporation into vesicles, although possible, is not the main reason. Rather, a rough estimate indicates that the NLS-semiconductor nanocrystals may have an overall size close to the nuclear pore sizes. In this case, intrinsic characteristics of the nuclear pores (size, shape, permeability, etc.) may become a dominant factor for the semiconductor nanocrystal entry. Indeed, a possible explanation invokes the variation of plasticity of the nuclear membrane during the cell cycle. The rate of nuclear pore formation of HeLa cells has been shown to vary with the cell cycle (Feldherr et al., J. Cell Biology, 111:1-8 (1990)). In addition, the newly formed pores comprise a subpopulation that are more permeable than mature ones. Because an asynchronous cells population was used in this study, we propose that HeLa cells with NLS-semiconductor nanocrystals in their nucleus represent a subpopulation of cells at a particular stage of the cell cycle.

### Example 4

### Observing Migratory Pathways of Semiconductor Nanocrystals

Cells were examined using the MRC-1024 laser scanning confocal microscope (Bio-Rad) with a Nikon Diaphot 200 microscope and a 60× PlanApo oil immersion objective lens (1.4 numerical aperture). Z-series (successive images collected by stepping through the cell at 0.2-0.5 μm intervals) were obtained to determine distribution of the semiconductor nanocrystals and time-series (same optical section imaged at 30-second intervals over time) were obtained to examine the dynamics of semiconductor nanocrystals in the cells. Semiconductor nanocrystals were excited using a krypton-argon laser at 488 nm. Cells were also examined using a Zeiss 510 NLO imaging system. Semiconductor nanocrystals were excited using an argon laser at 488 nm or Ti-Sapphire laser at 760 nm.

The visualization of the routes of semiconductor nanocrystal movement provide a means to better understand the protein nuclear trafficking process mediated by the nuclear localization signal, in this case, the SV40 NLS. While images of RP-semiconductor nanocrystals are mainly static in time, the fact that NLS-semiconductor nanocrystals actively seek to enter the nucleus can be visualized by tracking them. The general direction of movements of the NLS-semiconductor nanocrystals goes from the periphery of the cytoplasm to the perinuclear region. However, at the nuclear membrane, some NLS-semiconductor nanocrystals remain stuck, while some apparently enter the nucleus. A qualitative feature of fluorescence distinguishes them. Some fluorescent spots are large and bright, photobrighten with time, and remain in close contact with the nuclear membrane. They are likely vesicles containing a large collection of NLS-semiconductor nanocrystals. Other spots are smaller and blurred, and weaker in intensity, but the intensity still is stable over time (yellow and white arrows). We associate these latter features to single NLS-semiconductor nanocrystals or to aggregates of a small number of NLS-semiconductor nanocrystals, since these are the features that are observed to enter the nucleus.

The NLS-semiconductor nanocrystals demonstrate extreme chemical stability and photostability in the cells. The fluorescence from NLS-semiconductor nanocrystal conjugates in the cells were measured as a function of time under continuous excitation. The electroporation did not decrease the NLS-semiconductor nanocrystal fluorescence, and in some cases, we even observed an increase in the signal. As mentioned previously, such photobrightening is observed for the brighter spots, which we tentatively attribute to vesicles or aggregates of semiconductor nanocrystals. In addition to short-term monitoring of individual cells, we also followed the transfected cells for a prolonged period, and the fluorescent signals from NLS-semiconductor nanocrystals were still present in the cells after a week.

### Example 5

### Cytotoxicity of Semiconductor Nanocrystal-Peptide Conjugates

In their elemental form, cadmium, selenium, zinc, and sulphur have all been known to cause acute and chronic toxicities in living organisms. The cellular toxicity of surfactant-stabilized CdSe semiconductor nanocrystals has been examined when they are introduced into the cytoplasm (Dubertret et al., Science, 298:1759-1762 (2002); Jaiswal et al., Nat Biotechnol., 21:47-51 (2003); Derfus et al., Nano Letters, 4:11-18 (2004)). However, it is the cell nucleus that contains the genetic material DNA and the transcriptional machinery of the cell, and these are more sensitive to permanent alterations and damages. No data yet exist on the toxicity of the NLS- and STV-semiconductor nanocrystals introduced into the nucleus. To demonstrate that the cells are not adversely affected by the NLS-semiconductor nanocrystals, we assayed for cytotoxicity by comparing the colony-forming capability of the transfected cells vs. sham-transfected cells (Cells transfected with NLS-qdot complexes at different concentrations or with PBS only were plated at density of 300 cells/60 mm dish and allowed to grow for 10 days. Colonies formed by individual cells were stained by Crystal Violet dye and counted by ColCount (Oxford Optronix, Oxford, UK), with the relative number of surviving colonies used as an index for cell vitality). The survival of the cells carrying different doses of semiconductor nanocrystals was compared with cells transfected with PBS only. The dose of semiconductor nanocrystals applied here are 100, 10, and 1 pmole/10⁶ cells. We found that the nuclear accumulation of NLS-semiconductor nanocrystals had minimal impact on cellular survival. This indicates that the silica coating of the semiconductor nanocrystals has successfully prevented the interaction of Cd, Se, Zn, and S with the proteins and DNA in the nucleus. The introduction of streptavidin and the SV40 nuclear localization signal, at the concentrations used in this study, had no adverse effects on cell survival. The cells transfected with NLS-semiconductor nanocrystals showed a statistically insignificant difference from the cells transfected with just PBS. Close to 100% of transfected cells survived in all the experiments, indicating that the cytotoxicity caused by the transfection procedure is negligible.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all Accession Nos., articles and references, including patent applications, patents and PCT publications, are incorporated herein by reference for all purposes.

What is claimed is:

1. A signal peptide-semiconductor nanocrystal conjugate comprising:
a semiconductor nanocrystal and a signal peptide attached to the nanocrystal to form a signal peptide-semiconductor nanocrystal conjugate, wherein the conjugate is less than about 20 nm in diameter.

2. The conjugate of claim 1, wherein the diameter of the signal peptide-semiconductor nanocrystal conjugate is about 5 to about 20 nm.

3. The conjugate of claim 1, wherein the diameter of the signal peptide-semiconductor nanocrystal conjugate is about 10 to about 15 nm.

4. The conjugate of claim 1, wherein the signal peptide is selected from the group consisting of: a nuclear-localizing signal peptide, a peroxisome-targeting signal peptide, a cell membrane-targeting signal peptide, a mitochondrial-targeting signal peptide, an endoplasmic reticulum-targeting signal peptide, and a trans-Golgi body-targeting signal peptide.

5. The conjugate of claim 1, wherein the signal peptide is a nuclear-localizing signal peptide.

6. The conjugate of claim 1, wherein the signal peptide comprises a sequence selected from the group consisting of: any of SEQ ID NOS:1-13.

7. The conjugate of claim 1, wherein the signal peptide comprises SEQ ID NO:1.

8. The conjugate of claim 1, wherein the semiconductor nanocrystal comprises a core and a shell around the core.

9. The conjugate of claim 8, wherein the core comprises two or more elements independently selected from the group consisting of: Group II elements, Group III elements, Group IV elements, Group V elements, Group VI elements, and combinations thereof.

10. The conjugate of claim 8, wherein the shell comprises at least one material selected from the group consisting of: MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, GaAs, InGaAs, InP, and InAs.

11. The conjugate of claim 8, wherein the shell further comprises a hydrophilic coating.

12. The conjugate of claim 11, wherein the hydrophilic coating is selected from the group consisting of: SiO, SiO₂, polyethylene glycol, an ether, a mercapto acid, and a hydrocarbonic acid.

13. The conjugate of claim 11, wherein
the core comprises CdSe; and
the shell comprises ZnS and SiO₂.

14. The conjugate of claim 11, wherein the semiconductor nanocrystal and the signal peptide are attached via a linking agent.

15. The conjugate of claim 11, further comprising a linking agent attached to the shell.

16. The conjugate of claim 15, wherein the linking agent is covalently bound to the shell.

17. The conjugate of claim 15, wherein the linking agent is selected from the group consisting of: a negatively charged moiety, a positively charged moiety and steric repulsion groups.

18. The conjugate of claim 15, wherein the linking agent is a thiol, amine, carboxyl, or polyethylene glycol.

19. The conjugate of claim 15, wherein the linking agent is a bifunctional crosslinker.

20. The conjugate of claim 19, wherein the bifunctional crosslinker comprises two reactive groups independently selected from the group consisting of: thiol, carboxylate, carbonyl, amine, hydroxyl, aldehyde, ketone, active hydrogen, ester, sulfhydryl and photoreactive moieties.

21. The conjugate of aim 1, wherein the semiconductor nanocrystal and the signal peptide are attached via a first linking agent and a second linking agent.

22. The conjugate of claim 14, wherein the seminconductor nanocrystal is bound to the first linking agent and the signal peptide is bound to the second linking agent.

23. The conjugate of claim 21, wherein the first linking agent and the second linking agent are independently selected from the group consisting of: avidin, streptavidin, biotin and a bifunctional crosslinker.

24. The conjugate of claim 21, wherein the first linking agent is biotin and the second linking agent is streptavidin.

25. A signal peptide-semiconductor nanocrystal conjugate comprising:
a semiconductor nanocrystal comprising a core and a shell, wherein the shell comprises a hydrophilic coating; and
a signal peptide attached to the nanocrystal via a linker to form a signal peptide-semiconductor nanocrystal conjugate, wherein the conjugate is less than about 20 nm in diameter.

26. The signal peptide-semiconductor nanocrystal conjugate of claim 25, wherein the signal peptide is attached to the nanocrystal via a linking agent.

27. A method for imaging cellular structures of a cell, comprising:
contacting the cell with the signal peptide-semiconductor nanocrystal conjugate of claim 1 under conditions such that the signal peptide-semiconductor nanocrystal conjugate is taken up by the cell; and
imaging the cell to track movement of the signal peptide-semiconductor nanocrystal conjugates within the cell.

28. The method of claim 27, wherein the cell is a live cell.

29. The method of claim 27, wherein the cell is contacted with the signal peptide-semiconductor nanocrystal conjugate for at least about 1 hour.

30. The method of claim 27, wherein the cell is contacted with the signal peptide-semiconductor nanocrystal conjugate for about 2 weeks.

31. The method of claim 27, wherein the cell is also subjected to electroporation.

32. The method of claim 31, wherein the electroporation occurs prior to contacting the cell with the signal peptide-semiconductor nanocrystal conjugate.

33. The method of claim 31, wherein the electroporation occurs after contacting the cell with the signal peptide-semiconductor nanocrystal conjugate.

34. A method for obtaining a population of labeled cells containing signal peptide-semiconductor nanocrystal conjugates, the method comprising:
contacting a cell with the signal peptide-semiconductor nanocrystal conjugate of claim 1 under conditions such that the signal peptide-semiconductor nanocrystal conjugate is taken up by the cell;
allowing the cell to divide at least once under conditions such that, following division, each cell contains at least one signal peptide-semiconductor nanocrystal conjugate, thereby generating a population of labeled cells containing signal peptide-semiconductor nanocrystal conjugates.

35. The method of claim 34, wherein the cell is contacted with the signal peptide-semiconductor nanocrystal conjugate for at least about 1 hour.

36. The method of claim 34, wherein the cell is contacted with the signal peptide-semiconductor nanocrystal conjugate for about 2 weeks.

37. The method of claim 34, wherein the cell is also subjected to electroporation.

38. The method of claim 37, wherein the electroporation occurs prior to contacting the cell with the signal peptide-semiconductor nanocrystal conjugate.

39. The method of claim 37, wherein the electroporation occurs after contacting the cell with the signal peptide-semiconductor nanocrystal conjugate.
